(19) United States
(12) Plant Patent Application Publication
(10) Pub. No.: US 2002/0194661 P1
(43) Pub. Date: Dec. 19, 2002

(54) Verbascum plant named 'Dijon'

(75) Inventor: Daniel M. Heims, Tigard, OR (US)

Correspondence Address: KLARQUIST SPARKMAN CAMPBELL, LEIGH & WHINSTON, LLP, One World Trade Center, Suite 1600, 121 S.W. Salmon Street, Portland, OR, 97204, US

(73) Assignee: Terra Nova Nurseries, Inc.

(21) Appl. No.: 09/885,235

(22) Filed: Jun. 19, 2001

(51) Int. Cl. A01H 5/00

(52) U.S. Cl. PLT/263

(57) ABSTRACT

A new and distinct Verbascum plant characterized by gold colored flowers with purple centers with good vigor.

The present invention relates to a new and distinct discovery of Verbascum, of the family Scrophulariaceae, which originated as a sport mutation from tissue culture of Verbascum ‘Helen Johnson’, an unpatented plant.

This new Verbascum variety has been reproduced only by asexual propagation. Each of the progeny exhibits identical characteristics to the original plant.

This plant is characterized by the following:

1. Gold flowers with purple centers.

2. Excellent vigor.

Asexual propagation by division and tissue culture as done in Canby, Oregon, shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

FIGURE 1 shows the whole plant of Verbascum ‘Dijon’ (foreground) in bloom in a test bed. FIG. 2 shows a close-up of the flowers.

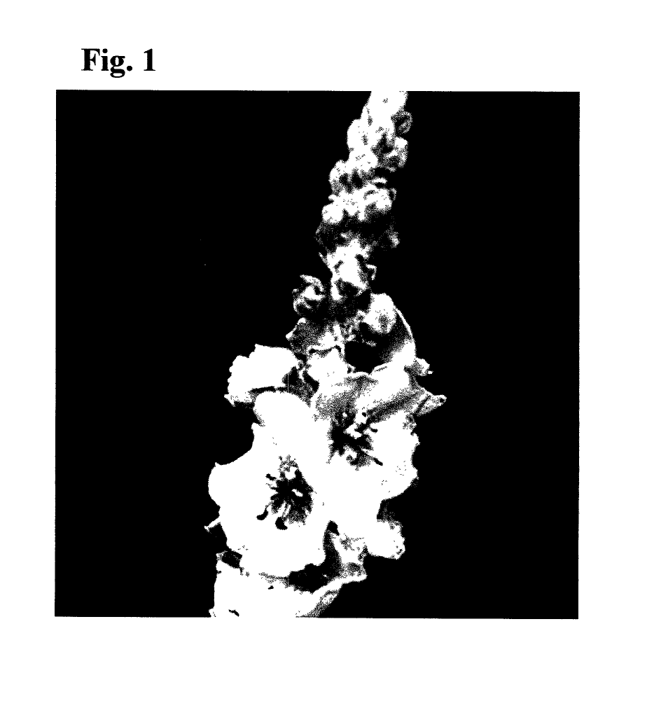

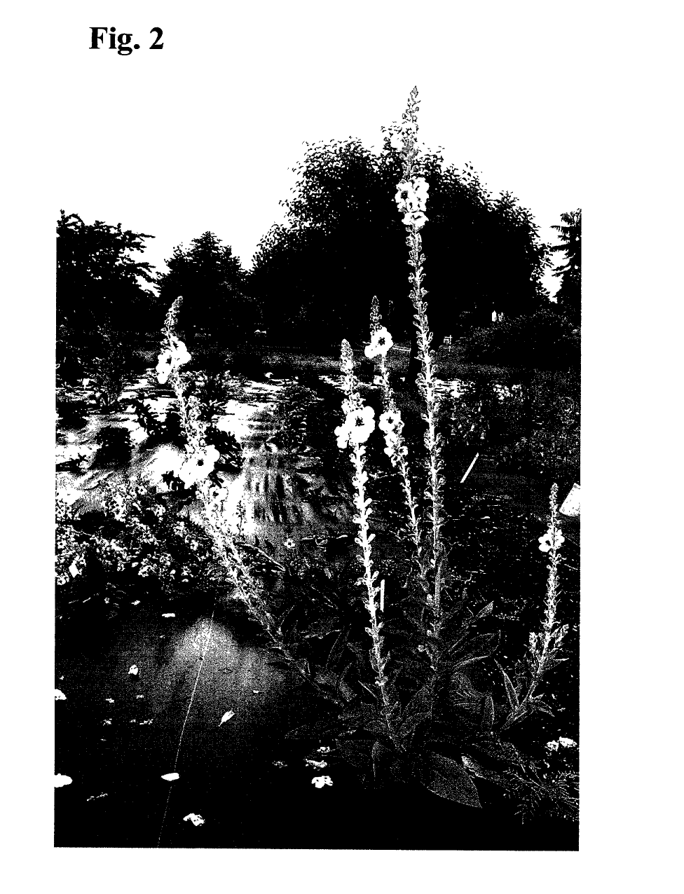

### DETAILED PLANT DESCRIPTION

The following is a detailed description of the new Verbascum selection based on observations of one-year-old specimens grown in the field in Canby, Oregon. The color descriptions are all based on The Royal Horticultural Society Colour Chart.

Botanical classification: Verbascum interspecific hybrid, species unknown, but probably the parents were V. chaixii and V. phoeniceum.

Plant:

Type.—Herbaceous perennial.

Form.—Basal rosette.

Hardiness.—U.S.D.A. Hardiness Zones 5-9.

Size.—About 85 cm tall and 60 cm wide, including inflorescence.

Leaf:

Type.—Simple. Evergreen.

Arrangement.—Alternate.

Shape.—Ovate.

Size.—Basal leaves grow to 18 cm. long and 8.5 cm. wide. Cauline leaves grow to 17.5 cm. long and 7.5 cm. wide.

Width.—Grows to 8.5 cm.

Venation.—Pinnate.

Margins.—Crenate.

Apex.—Acute.

Base.—Oblique to truncate.

Texture.—Thick, leathery, rugose, soft, pubescent.

Petiole: Length—0 to 0.5 cm Texture.—Tomentose. Color - Yellow Green 145B.

Color.—Topside—Deep green. Green 137A with lighter main vein, Yellow Green 145D or sometimes pink, Greyed Red 182C. Bottomside - Closest to Yellow Green 147B.

Inflorescence:

Type.—Raceme, branching on bottom half.

Flower number.—About 100 per raceme before branching.

Peduncle.—Height—82 cm. Diameter—0.9 cm. Texture—Soft with white tomentose hairs. Color—Yellow Green 144A with heavy white tomentose hairs.

Pedicel.—Length—Grows to 0.3 cm. Texture—Covered with white tomentose hairs. Color—Under hairs it is Yellow Green 144A.

Bloomtime.—May to August in Canby, Oreg.

Flower Bud:

Size.—1 cm. wide and 0.8 cm. deep.

Description.—Round or pentagonal cushion.

Surface.—Sparsely pubescent.

Color.—Greyed Orange 164B with Greyed Red 178A to B on petal edges.

Flower:

Type.—Perfect. Complete. Irregular.

Shape.—Rotate, irregular, short tubular.

Size.—4 cm wide and 0.4 cm deep.

Color.—Yellow Orange 14C to Yellow 11B with Greyed Purple 184A streaks to center. The center is Purple 77A.

Corolla.—Petal number—5. Size—2 cm long and 2 cm wide. Surface—Backs sparsely pubescent

Calyx.—Sepal number—5 Size—Grows to 1 cm. deep and 0.8 cm. wide. Texture—White tomentose. Color—Yellow Green 138A under hairs.

Stamens.—Number—5 Filaments—0.7 cm. long, Greyed Orange 164B in color and heavily bearded Purple 77A. Anthers—Kidney shaped. Greyed Orange 171A to 172A. Pollen—None produced.

Fragrance.—none.

Fertility: Sterile, male and female.

Disease: None observed on plants grown commercially in Canby, Oreg.

### COMPARISONS

The instant plant resulted from a discovery made by the applicant of a Verbascum ‘Helen Johnson’ sport. Verbascum ‘Dijon’ exhibits the same great characteristics of Verbascum ‘Helen Johnson’. These are vigor, strong stems, bold foliage, and sterility. The significant difference is the flower color. V. ‘Helen Johnson’ has buff-apricot colored flowers with purple centers, while this new plant has gold colored flowers with purple center.

I claim:

1. A new and distinct variety of Verbascum plant substantially as shown and described, characterized by a unique gold flowers with purple centers.
